(19) United States
(12) Patent Application Publication
(10) Pub. No.: US 2019/0382369 A1
(43) Pub. Date: Dec. 19, 2019

(54) 4-((6-(2-(2,4-DIFLUOROPHENYL)-1,1-DIFLUORO-2-HYDROXY-3-(1H-1,2,4-TRIAZOL-1-YL)PROPYL)PYRIDIN-3-YL)OXY)BENZONITRILE AND PROCESSES OF PREPARATION

(71) Applicant: Dow AgroSciences LLC, Indianapolis, IN (US)

(72) Inventors: Qiang Yang, Zionsville, IN (US); Yan Hao, Zionsville, IN (US); Sarah Ryan, Indianapolis, IN (US); Gregory Whiteker, Carmel, IN (US)

(21) Appl. No.: 16/455,005

(22) Filed: Jun. 27, 2019

Related U.S. Application Data
(63) Continuation of application No. 15/776,696, filed on May 16, 2018, now abandoned.
(60) Provisional application No. 62/256,531, filed on Nov. 17, 2015.

(51) Int. Cl. C07D 401/06

(52) U.S. Cl. CPC C07D 401/06; C07D 249/08

(57) ABSTRACT

Provided herein is a process for the preparation of 4-((6-(2-(2,4-difluorophenyl)-1, 1-difluoro-2-hydroxy-3-(1H-1,2,4-triazol-1-yl)propyl)pyridin-3-yl)oxy)benzonitrile.

### CROSS-REFERENCE TO RELATED APPLICATIONS

This application claims priority to U.S. Provisional Application No. 62/256,531, filed Nov. 17, 2015, which is incorporated herein by reference in its entirety.

### FIELD

Provided herein is 4-((6-(2-(2,4-difluorophenyl)-1,1-difluoro-2-hydroxy-3-(1H-1,2,4-triazol-1-yl)propyl)pyridin-3-yl)oxy)benzonitrile and processes of preparation.

### BACKGROUND

U.S. patent application Ser. Nos. 13/527,387, 13/527,426 and 13/528,283 describe inter alia certain metalloenzyme inhibitor compounds and their use as fungicides. The disclosure of each application is expressly incorporated by reference herein. Each of these patent applications describe various routes to generate metalloenzyme inhibiting fungicides. It may be advantageous to provide more direct and efficient methods for the preparation of metalloenzyme inhibiting fungicides and related compounds, e.g., by the use of reagents and/or chemical intermediates which provide improved time and cost efficiency.

### SUMMARY OF THE DISCLOSURE

Provided herein is the compound 4-((6-(2-(2,4-difluorophenyl)-1,1-difluoro-2-hydroxy-3-(1H-1,2,4-triazol-1-yl)propyl)pyridin-3-yl)oxy)benzonitrile (I) and processes for its preparation. In one embodiment, provided herein, is a process for the preparation of the compound of the Formula I:

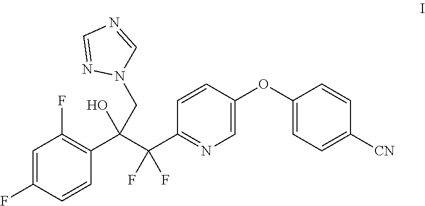 [SMILES: N#Cc1ccc(Oc2ccc(C(F)(F)C(O)(Cn3cncn3)c3ccc(F)cc3F)nc2)cc1]

which comprises contacting a compound of Formula II with a trialkylsulfoxonium halide, a base, and 1H-1,2,4-triazole.

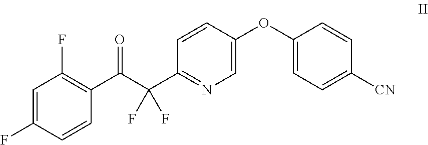 [SMILES: N#Cc1ccc(Oc2ccc(C(F)(F)C(=O)c3ccc(F)cc3F)nc2)cc1]

In another embodiment, the compound of Formula II may be prepared by contacting a compound of Formula III

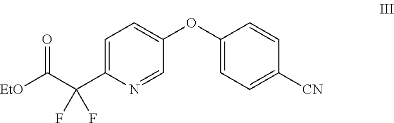 [SMILES: CCOC(=O)C(F)(F)c1ccc(Oc2ccc(C#N)cc2)cn1]

with a mixture formed by combining 1-bromo-2,4-difluorobenzene with a metal or an organometallic reagent, and an acid.

In another embodiment, the compound of Formula III may be prepared by contacting a compound of Formula IV with ethyl 2-bromo-2,2-difluoroacetate and a metal.

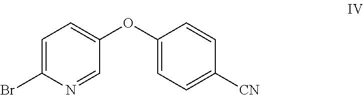 [SMILES: N#Cc1ccc(Oc2ccc(Br)nc2)cc1]

In another embodiment, the compound of Formula IV may be prepared by contacting a compound of Formula V with 4-fluorobenzonitrile or 4-nitrobenzonitrile, and a base.

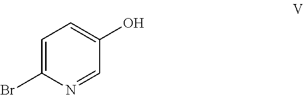

In another embodiment, the compound of Formula V may be prepared by contacting a compound of Formula VI with a magnesium-halogen exchange reagent, a borate, and an oxidizing agent.

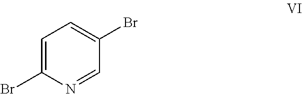

The term “hydroxyl” refers to an —OH substituent.

The term “halogen” or “halo” refers to one or more halogen atoms, defined as F, Cl, Br, and I.

The term “organometallic” refers to an organic compound containing a metal, especially a compound in which a metal atom is bonded directly to a carbon atom.

Room temperature (RT) is defined herein as about 20° C. to about 25° C.

Throughout the disclosure, references to the compounds of Formula I is read as also including optical isomers and salts. Specifically, when compounds of Formula I contain a chiral carbon, it is understood that such compounds include optical isomers and racemates thereof. Exemplary salts may include: hydrochloride, hydrobromide, hydroiodide, and the like.

Certain compounds disclosed in this document can exist as one or more isomers. It will be appreciated by those skilled in the art that one isomer may be more active than the others. The structures disclosed in the present disclosure are drawn in only one geometric form for clarity, but are intended to represent all geometric and tautomeric forms of the molecule.

The embodiments described above are intended merely to be exemplary, and those skilled in the art will recognize, or will be able to ascertain using no more than routine experimentation, numerous equivalents of specific processes, materials and procedures. All such equivalents are considered to be within the scope of the invention and are encompassed by the appended claims.

### DETAILED DESCRIPTION

4-((6-(2-(2,4-Difluorophenyl)-1,1-difluoro-2-hydroxy-3-(1H-1,2,4-triazol-1-yl)propyl)pyridin-3-yl)oxy)benzonitrile (I) is provided herein and may be prepared from 2,5-dibromopyridine (VI) as shown in Examples 1-5.

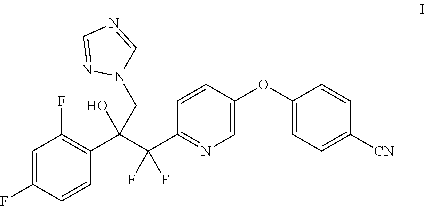 [SMILES: N#Cc1ccc(Oc2ccc(C(F)(F)C(O)(Cn3cncn3)c3ccc(F)cc3F)nc2)cc1]

### Example 1: Preparation of 6-bromopyridin-3-ol (V)

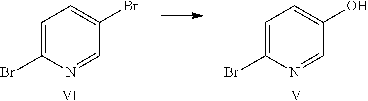

2,5-Dibromopyridine (VI) (9.98 g, 42.1 mmol) was dissolved in 53 mL anhydrous THF under nitrogen in a 250 mL 3-neck flask equipped with a mechanical stirrer, a thermocouple and a nitrogen inlet. A light tan solution was formed. A 2 M solution of i-PrMgCl in ether (23 mL) was added via syringe over 3 min. When approximately 50% of the Grignard solution had been added, a brown suspension formed. Addition of i-PrMgCl caused an exotherm to 36° C. After stirring for 90 min, the suspension was cooled to 2° C., and neat trimethylborate (B(OMe)₃) was added rapidly via syringe. The reaction exothermed to 6° C., and the ice bath was removed. After stirring overnight, glacial acetic acid (3.79 g) was added, causing all solids to dissolve and a dark brown solution to form. The solution was cooled in an ice bath and 5.25 g of 30% hydrogen peroxide (an oxidizing agent) was added dropwise at a rate which kept the reaction temperature from exceeding 12° C. The reaction mixture was stirred for 90 min, and then diethyl ether (150 mL) and water (100 mL) were added. The aqueous layer was separated and extracted with ether (2×100 mL). The combined organics were washed with a 100 mL 10% sodium bisulfite solution and then brine. The extracts were dried (MgSO₄) and rotary evaporated to a brown oil which formed a tan solid on standing (7.95 g). The crude product was adsorbed onto 15 g Celite® and purified by flash chromatography using a 220 g silica column and hexanes/EtOAc gradient. Fractions were evaporated to give 4.81 g (66% yield) of an off-white solid. NMR spectra were identical to that of an authentic sample of 6-bromo-3-pyridinol. ¹H NMR (DMSO-d₆, 400 mHz) δ 10.24 (s, 1H), 7.94 (d, J=3.0 Hz, 1H), 7.42 (d, J=8.6 Hz, 1H), 7.17 (dd, J=3.0, 8.6 Hz, 1H); ¹³C NMR (DMSO-d₆, 101 MHz) δ 153.74, 138.13, 129.30, 128.14, 126.21.

The process exemplified in Example 1 may be conducted with additional Grignard reagents, such as, for example, EtMgX, MeMgX, i-PrMgX, n-BuMgX, or PhMgX, wherein X is Cl or Br. The described process may also be conducted with a Grignard reagent, such as, for example, n-BuMgX, in the presence of a metal-halogen exchange reagent, such as, for example, n-BuLi. The described process may also be conducted with alternative borates, such as, for example, B(OEt)₃ or B(Oi-Pr)₃. Solvents for use in this process may include those selected from THF, 2-MeTHF, MTBE, and dioxane.

The oxidizing agent used in the process exemplified in Example 1 may be selected from the group including hydrogen peroxide, peracetic acid, and a mixture of hydrogen peroxide and acetic acid.

### Example 2: Preparation of 4-((6-bromopyridin-3-yl)oxy)benzonitrile (IV)

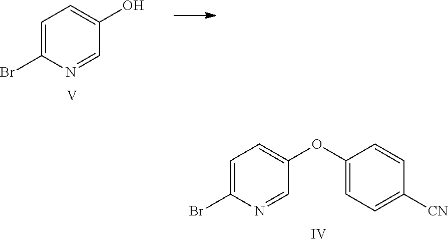

### Method A:

To a 250-mL flask were charged 6-bromopyridin-3-ol (V) (10 g, 57.5 mmol), 4-fluorobenzonitrile (8.35 g, 69.0 mmol), potassium carbonate (15.89 g, 115 mmol), and DMF (50 mL). The reaction was heated at 90° C. for 20 h, at which point HPLC analysis indicated that the reaction was complete. The reaction mixture was allowed to cool to 20° C., and then was further cooled to 0° C. Water (150 mL) was added, while maintaining the internal temperature at less than 15° C. (exotherm during the addition of water). The resulting suspension was stirred at 20° C. for 1 h and filtered. The filter cake was rinsed with water (2×25 mL) to afford a white solid. The solid was suspended in 95% ethanol (65 mL) and heated to 75° C. to afford a clear solution. It was allowed to cool to 20° C. over 1 h, and the resulting white suspension was stirred at 20° C. for 2 h. The suspension was filtered, and the solid was rinsed with 95% ethanol (2×10 mL). The solid was dried under vacuum to afford the desired product as a white solid (13.2 g, 83% yield). ¹H NMR (400 MHz, CDCl₃) δ 8.22 (d, J=3.0 Hz, 1H), 7.73-7.63 (m, 2H), 7.53 (d, J=8.6 Hz, 1H), 7.33-7.23 (m, 1H), 7.14-7.00 (m, 2H); ¹³C NMR (101 MHz, CDCl₃) δ 160.13, 151.47, 142.54, 136.81, 134.47, 130.10, 129.12, 118.33, 118.23, 107.56; ESIMS: m/z 277.1 ([M+H]⁺).

### Method B:

To a 250-mL round bottom flask were charged 6-bromopyridin-3-ol (V) (10 g, 57.5 mmol), 4-nitrobenzonitrile (8.94 g, 60.3 mmol), potassium carbonate (15.9 g, 114.9 mmol), and DMF (30 mL). The reaction was heated at 90° C. for 18 h, at which point HPLC analysis indicated that the reaction was complete. The reaction was allowed to cool to 20° C. and diluted with water (90 mL) at less than 50° C. The resulting suspension was stirred for 1 h and filtered. The filter cake was rinsed with water (2×50 mL) to give an off-white solid. The resulting solid was suspended in EtOH (40 mL) and heated to 75° C. to afford a clear solution. It was allowed to cool to 20° C. over 2 h, and stirred at this temperature for 1 h. The resulting suspension was filtered and the filter cake was rinsed with EtOH (2×10 mL). The filter cake was dried to afford the desired product as a white solid (12.9 g, 82% yield). mp: 116-119° C. ¹H NMR (400 MHz, CDCl₃) δ 8.22 (d, J=3.0 Hz, 1H), 7.67 (d, J=8.8 Hz, 2H), 7.53 (d, J=8.6 Hz, 1H), 7.29 (dd, J=8.7, 2.9 Hz, 1H), 7.07 (d, J=8.8 Hz, 2H). ¹³C NMR (101 MHz, CDCl₃) δ 160.13, 151.47, 142.55, 136.81, 134.48, 130.13, 129.13, 118.34, 107.55. ESIMS: m/z 277.0 ([M+H]⁺).

The process exemplified in Example 2 may be conducted in a solvent selected from one or more of dimethyl sulfoxide (DMSO), dimethylacetamide (DMA), dimethylformamide (DMF), and N-methyl-2-pyrrolidone (NMP). Bases for use in this process may include metal carbonates such as potassium carbonate and cesium carbonate, metal hydrides such as NaH, metal hydroxides such as NaOH and KOH, and metal bicarbonates.

The process exemplified in Example 2 may be conducted between about room temperature and about 120° C.

### Example 3: Preparation of ethyl 2-(5-(4-cyanophenoxy)pyridin-2-yl)-2,2-difluoroacetate (III)

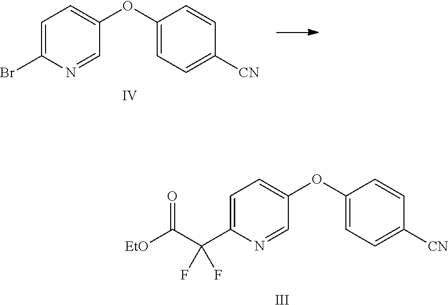

### Method A:

Ethyl 2-bromo-2,2-difluoroacetate (12.27 mL, 94 mmol) and copper powder (14-25 μm, 9.60 g, 151 mmol) were added to a solution of 4-((6-bromopyridin-3-yl)oxy)benzonitrile (IV) (20 g, 72.0 mmol) in DMF (140 mL) under nitrogen. The resulting brown suspension was heated at 60° C. under nitrogen for 18 h, at which point HPLC analysis indicated that the reaction was complete. The mixture was cooled to 20° C., and MTBE (280 mL) was added. The resulting mixture was stirred for 10 min and filtered through a Celite® pad. The Celite® pad was rinsed with MTBE (2×140 mL). The filtrate was washed with sat. NH₄Cl (200 mL), brine (3×140 mL), and water (2×140 mL). The organic layer was dried over anhydrous Na₂SO₄, filtered, and concentrated to afford the crude product as a light brown oil (21 g, 92%) in purity sufficient for use in the next step directly. This crude product was further purified by column chromatography (10-20% EtOAc/hexanes) to give the desired product as a white solid (16 g, 70% yield); mp 45-48° C. ¹H NMR (400 MHz, CDCl₃) δ 8.44 (d, J=2.7 Hz, 1H), 7.79 (dd, J=8.6, 0.7 Hz, 1H), 7.73-7.66 (m, 2H), 7.49 (dd, J=8.6, 2.7 Hz, 1H), 7.14-7.08 (m, 2H), 4.40 (q, J=7.1 Hz, 2H), 1.36 (t, J=7.1 Hz, 3H); ESIMS m/z 319.1 ([M+H]⁺).

### Method B:

To a 15 L jacketed reactor were added 4-((6-bromopyridin-3-yl)oxy)benzonitrile (IV) (900 g, 3173 mmol), ethyl 2-bromo-2,2-difluoroacetate (541 mL, 4125 mmol), copper (423 g, 6664 mmol), and DMSO (4500 mL) under nitrogen to give a brown suspension. The reaction was heated at 40° C. for 8 h, at which point HPLC analysis indicated that the reaction was complete. It was allowed to cool to 20° C. and MTBE (4000 mL) was added. The mixture was stirred for 30 minutes and filtered through a Celite® pad. The filter pad was rinsed with MTBE (2×1000 mL) and the combined filtrates were rinsed with brine (3×2000 mL). The first aqueous layer was extracted with MTBE (2×1000 mL). The combined organic layers were washed with a saturated NH₄Cl solution (2×2000 mL) and brine (3×2000 mL), and concentrated to give the desired product as a brown oil (1030 g, 96% yield). ¹H NMR (400 MHz, CDCl₃) δ 8.44 (d, J=2.7 Hz, 1H), 7.79 (dd, J=8.6, 0.7 Hz, 1H), 7.73-7.66 (m, 2H), 7.49 (dd, J=8.6, 2.7 Hz, 1H), 7.14-7.08 (m, 2H), 4.40 (q, J=7.1 Hz, 2H), 1.36 (t, J=7.1 Hz, 3H).

The process exemplified in Example 3 may be conducted in a solvent selected from one or more of DMSO, DMF, THF, and NMP, and with a metal such as copper.

The process exemplified in Example 3 may be conducted between about room temperature and about 100° C.

### Example 4: Preparation of 4-((6-(2-(2,4-difluorophenyl)-1,1-difluoro-2-oxoethyl)pyridin-3-yl)oxy)benzonitrile (II)

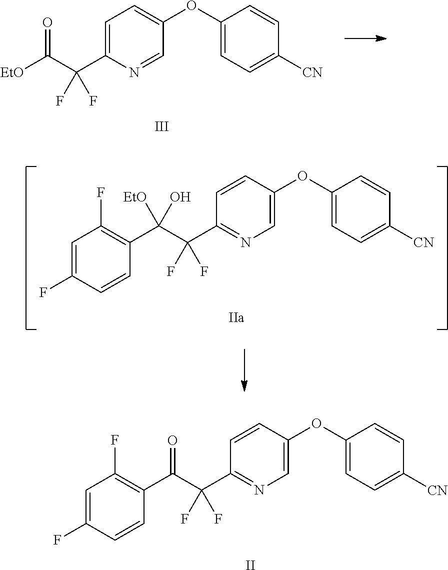

### Method A:

A suspension of Mg turnings (3.47 g, 143 mmol) in THF (250 mL) was heated to 35° C. under nitrogen. A portion of 1-bromo-2,4-difluorobenzene (1 mL, 8.85 mmol) was added to the reactor, and the resulting mixture was heated at 35° C. for 30 min to initiate the reaction. The reaction mixture was cooled to 30° C., and the remainder of 1-bromo-2,4-difluorobenzene (16.4 mL, 145.15 mmol) was added to the reactor at 28-32° C. over 30 min. The reaction was stirred at 30° C. for 2 h, at which point complete consumption of Mg was observed. The reaction was cooled to less than 0° C., and a solution of ethyl 2-(5-(4-cyanophenoxy)pyridin-2-yl)-2,2-difluoroacetate (III) (35 g, 110 mmol) in THF (100 mL) was added at less than 5° C. over 30 min. The reaction was stirred at 0° C. for 1 h and quenched into a 2 N HCl solution (150 mL) at less than 10° C. (pH=1-2). The reaction was stirred at 20° C. for 18 h, at which point HPLC analysis indicated that there was still about 10% of hemiketal intermediate (IIa) remaining. It was further stirred at 30° C. for 5 h, at which point HPLC analysis indicated that the hemiketal (IIa) intermediate was fully consumed. The layers were separated, and the aqueous layer was extracted with EtOAc (100 mL). The combined organic layers were washed with a sat. NaHCO₃ solution (100 mL), dried over anhydrous Na₂SO₄, filtered, and concentrated to give a light tan solid (45.6 g). The solid was dissolved in EtOAc (60 mL) at 60° C., and heptane (100 mL) was added. The mixture was seeded and stirred at 20° C. for 18 h to afford a suspension. The suspension was filtered and the solid was dried to afford the desired product as a white solid (25.5 g). The filtrate was concentrated and recrystallized from MTBE (50 mL) and heptane (100 mL) to give a light brown solid (14.1 g) after drying, affording a combined yield of 90%. ¹H NMR (400 MHz, CDCl₃) δ 8.37 (d, J=2.7 Hz, 1H), 8.08 (td, J=8.4, 6.4 Hz, 1H), 7.87 (d, J=8.6 Hz, 1H), 7.75-7.66 (m, 2H), 7.54 (dd, J=8.6, 2.8 Hz, 1H), 7.17-7.08 (m, 2H), 7.01 (dddd, J=8.6, 7.6, 2.5, 0.9 Hz, 1H), 6.84 (ddd, J=11.0, 8.6, 2.4 Hz, 1H); ESIMS m/z 387.0 ([M+H]⁺).

### Method B:

A suspension of Mg turnings (107 g, 4.3 mol) in THF (6000 mL) was heated to 35° C. under nitrogen. A portion of 1-bromo-2,4-difluorobenzene (32 mL, 0.28 mol) was added to the reactor at 35° C., and the resulting mixture was heated at 35° C. for 30 min to initiate the reaction. The reaction mixture was cooled to 15° C., and the remainder of 1-bromo-2,4-difluorobenzene (500 mL, 4.45 mol) was added to the reactor at 15-20° C. over 80 min. The reaction was stirred at 20° C. for 1 h and cooled to −20° C. A solution of ethyl 2-(5-(4-cyanophenoxy)pyridin-2-yl)-2,2-difluoroacetate (III) (1052 g, 3.07 mol) in THF (100 mL) was added at less than −5° C. over 40 min. The container and addition funnel were rinsed with THF (200 mL) and the rinse solvent was added to the reaction. The reaction was stirred at −20° C. for 2 h and quenched into a 4 N HCl solution (1500 mL) at less than 10° C. The reaction was allowed to warm to 20° C. and stirred for 16 h, at which point HPLC analysis indicated that the reaction was complete. The layers were separated, and the aqueous layer was extracted with MTBE (3×400 mL). The combined organic layers were washed with a saturated NaHCO₃ solution (2×1000 mL), brine (2×1000 mL), and water (1000 mL). The organic layer was dried, filtered, and concentrated to afford a brown solid (1264 g). The resulting solid was suspended in 3:1 heptane/MTBE (1000 mL) and heated at 60° C. for 1 h. The resulting suspension was cooled to ambient temperature and filtered. The solid was suspended in 3:1 heptane/MTBE (1000 mL) and heated at 60° C. for 1 h. The resulting suspension was cooled to ambient temperature and filtered to give the desired product as a tan solid after drying (1080 g, 86% yield). Analysis of the isolated product was in agreement with that of the previously obtained sample.

The process exemplified in Example 4 (Methods A and B) may be conducted in a solvent that is an aprotic solvent selected from one or more of diethyl ether, tetrahydrofuran (THF), 1,2-dimethoxyethane (DME), toluene, dioxane and methyl t-butyl ether (MTBE).

The process exemplified in Example 4 (Methods A and B) may be conducted with an organometallic reagent that is either an aryl Grignard or an aryl lithium reagent formed by a reaction of 2,4-difluoro-1-bromobenzene with one of magnesium, an alkyllithium reagent such as n-butyllithium, or a Grignard reagent such as isopropylmagnesium chloride.

The process exemplified in Example 4 (Methods A and B) may be conducted between about −80° C. and about 50° C.

The hemiketal of Formula IIa may be isolated as an intermediate in the process to prepare the compound of Formula II under certain reaction conditions (e.g., see Method C). Addition of a acid to the hemiketal of Formula IIa (e.g., see Method D) or heating it at elevated temperature (e.g., see Method E) results in conversion of it into the desired product of Formula II.

Suitable acids for use in the process exemplified in Example 4 (Methods A-D) may include HCl, HBr, H₂SO₄, H₃PO₄, HNO₃, acetic acid, trifluoroacetic acid, and mixtures thereof.

### Method C:

### Preparation of 4-((6-(2-(2,4-difluorophenyl)-2-ethoxy-1,1-difluoro-2-hydroxyethyl)pyridin-3-yl)oxy)benzonitrile (IIa)

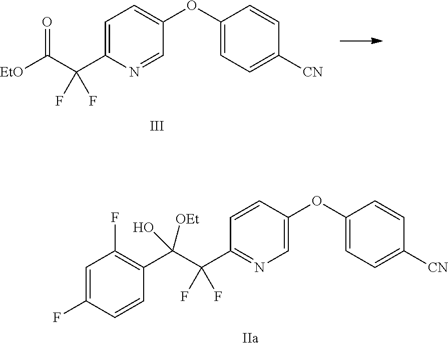

A suspension of Mg turnings (0.458 g, 18.85 mmol) in THF (25 mL) was heated to 35° C. under nitrogen. A portion of 1-bromo-2,4-difluorobenzene (0.25 mL, 2.99 mmol) was added to the reactor, and the resulting mixture was heated at 35° C. for 30 min to initiate the reaction. The reaction mixture was cooled to 30° C., and the remainder of 1-bromo-2,4-difluorobenzene (1.46 mL, 17.43 mmol) was added to the reactor at less than 35° C. The reaction was stirred at 30° C. for 2 h, at which point complete consumption of Mg was observed. The reaction was cooled to less than 0° C., and a solution of ethyl 2-(5-(4-cyanophenoxy)pyridin-2-yl)-2,2-difluoroacetate (II) (5.0 g, 15.71 mmol) in THF (25 mL) was added at less than 5° C. The reaction was stirred at 0° C. for 1 h and quenched into a 2 N HCl solution (24 mL) at less than 10° C. The reaction mixture was diluted with water (30 mL) and extracted with EtOAc (50 mL). The organic layer was concentrated to give a semi-solid. The crude product was dissolved in EtOAc (5 mL) with heating and heptane (40 mL) was added over 15 min to give a yellow suspension. The mixture was stirred at 20° C. for 1 h and filtered. The solid was rinsed with heptane (2×10 mL) and air-dried to afford the desired product as a yellow solid (5.1 g, 75% yield). ¹H NMR (400 MHz, CDCl₃) δ 8.43 (d, J=2.7 Hz, 1H), 7.89-7.77 (m, 2H), 7.75-7.67 (m, 2H), 7.59-7.49 (m, 1H), 7.25 (s, 1H), 7.17-7.10 (m, 2H), 6.95 (tdd, J=8.7, 2.6, 0.9 Hz, 1H), 6.85 (ddd, J=11.4, 8.9, 2.6 Hz, 1H), 3.66 (dq, J=9.6, 7.1 Hz, 1H), 3.33 (dq, J=9.6, 7.0 Hz, 1H), 1.04 (t, J=7.1 Hz, 3H); ESIMS in& 433.1 ([M+H]⁺).

### Method D:

### Preparation of 4-((6-(2-(2,4-difluorophenyl)-1,1-difluoro-2-oxoethyl)pyridin-3-yl)oxy)benzonitrile (II)

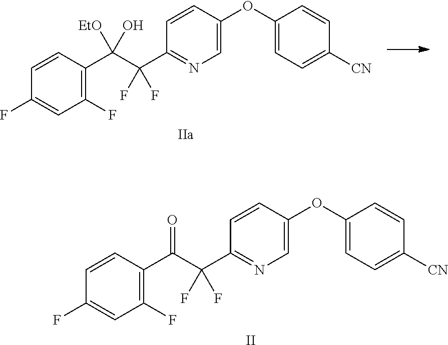 [SMILES: CCOC(O)(c1ccc(F)cc1F)C(F)(F)c1ccc(Oc2ccc(C#N)cc2)cn1 ; "IIa" ; N#Cc1ccc(Oc2ccc(C(F)(F)C(=O)c3ccc(F)cc3F)nc2)cc1 ; "II"]

A sample of 4-((6-(2-(2,4-difluorophenyl)-2-ethoxy-1,1-difluoro-2-hydroxyethyl)pyridin-3-yl)oxy)benzonitrile (IIa) (200 mg, 0.463 mmol) was dissolved in 2 N HCl (1 mL) and THF (2 mL) and was stirred at 20° C. for 18 h. It was neutralized with NaHCO₃ to pH 6-7 and extracted with EtOAc. The organic layer was concentrated to dryness to afford the desired product as a yellow oil. Analytical data of the isolated product were consistent with that of previously obtained samples.

### Method E

### Preparation of 4-((6-(2-(2,4-difluorophenyl)-1,1-difluoro-2-oxoethyl)pyridin-3-yl)oxy)benzonitrile (I)

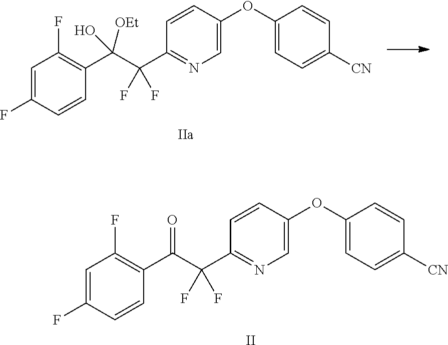 [SMILES: CCOC(O)(c1ccc(F)cc1F)C(F)(F)c1ccc(Oc2ccc(C#N)cc2)cn1 ; "IIa" ; N#Cc1ccc(Oc2ccc(C(F)(F)C(=O)c3ccc(F)cc3F)nc2)cc1 ; "II"]

A sample of 4-((6-(2-(2,4-difluorophenyl)-2-ethoxy-1,1-difluoro-2-hydroxyethyl)pyridin-3-yl)oxy)benzonitrile (IIa) (8.8 g, 20.35 mmol) was suspended in toluene (30 mL) and heated at 105° C. for 8 h. It was cooled to 20° C. and concentrated under reduced pressure to afford a yellow oil. The residue was dissolved in EtOAc (8 mL) and heptane (64 mL) was added. The mixture was stirred for 2 h and filtered. The filter cake was rinsed with heptanes (2×20 mL) and dried to afford a light yellow solid (5.8 g, 74% yield). Analytical data of the isolated product (II) were consistent with that of previously obtained samples.

### Example 5: Preparation of 4-((6-(2-(2,4-difluorophenyl)-1,1-difluoro-2-hydroxy-3-(1H-1,2,4-triazol-1-yl)propyl)pyridin-3-yl)oxy)benzonitrile (I)

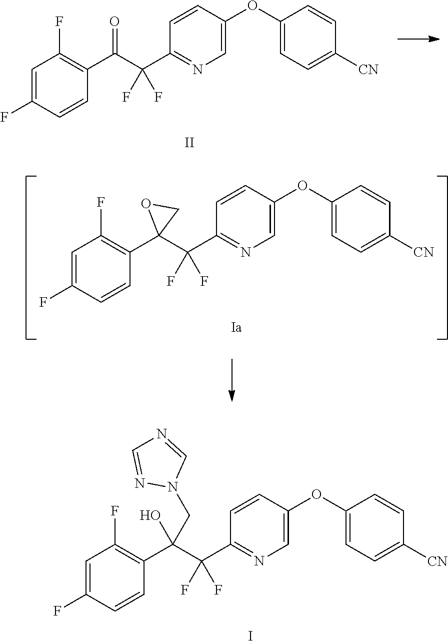

### Method A:

Potassium carbonate (32.6 g, 236 mmol) was charged to a suspension of trimethylsulfoxonium iodide (26.5 g, 118 mmol) in NMP (190 mL) at less than 5° C., and the reaction was stirred at 20° C. for 2 h to give a white suspension. 4-((6-(2-(2,4-Difluorophenyl)-1,1-difluoro-2-oxoethyl)pyridin-3-yl)oxy)benzonitrile (II) (38 g, 94 mmol) was added in one portion, and the reaction was stirred at 35° C. under N₂ for 18 h, at which point HPLC analysis indicated that the starting material was fully converted to the epoxide intermediate (Ia). 1H-1,2,4-Triazole (8.56 g, 123 mmol) was added, and the reaction was stirred at 60° C. for 18 h, at which point HPLC analysis showed about 10% epoxide intermediate (Ia) remaining. The reaction was further stirred at 80° C. for 1 h, at which point HPLC analysis indicated that the reaction was complete. The mixture was allowed to cool to 20° C. and was poured into ice water (1200 mL). The resulting suspension was filtered, and the solid was dissolved in DCM (1200 mL). The solution was washed with brine (2×300 mL) and the organic layer was concentrated to about 200 mL. The resulting solution was purified by column chromatography (750 g silica) using EtOAc/hexanes as eluent to afford the desire product as a light yellow foam (39.2 g, 85% yield). ¹H NMR (400 MHz, CDCl₃) δ 8.36 (d, J=2.7 Hz, 1H), 8.15 (d, J=1.0 Hz, 1H), 7.74 (s, 1H), 7.73-7.67 (m, 2H), 7.58 (dd, J=8.7, 0.6 Hz, 1H), 7.51-7.44 (m, 1H), 7.42 (dd, J=8.7, 2.8 Hz, 1H), 7.15-7.03 (m, 2H), 6.81-6.68 (m, 2H), 6.27 (s, 1H), 5.40 (d, J=14.4 Hz, 1H), 4.93-4.82 (m, 1H); ESIMS m/z 470.0 ([M+H]⁺).

### Method B:

To a 100-mL, 3-neck, round bottom flask were charged trimethylsulfoxonium iodide (0.356 g, 1.618 mmol) and NMP (5 mL). NaOt-Bu (0.143 g, 1.488 mmol) was added at less than 25° C., and the reaction was stirred at 20° C. for 1 h. The reaction was cooled to less than −15° C. and 4-((6-(2-(2,4-difluorophenyl)-1,1-difluoro-2-oxoethyl)pyridin-3-yl)oxy)benzonitrile (II) (0.5 g, 1.294 mmol) was added. The reaction was stirred at less than −10° C. for 1 h, after which time HPLC analysis indicated that the starting material had been fully converted to the epoxide intermediate (Ia). 1H-1,2,4-Triazole (0.103 g, 1.488 mmol) and NaOt-Bu (0.143 g, 1.488 mmol) were added, and the reaction was heated at 40° C. for 6 h. The reaction was cooled to 20° C. and added with water (20 mL). The mixture was extracted with EtOAc (2×20 mL). The organics were concentrated to dryness and purified by column chromatography (40 g silica, 0-60% EtOAc/hexanes over 5 column volumes, hold for 5 volumes). Fractions containing pure product were concentrated to afford a colorless oil (400 mg, 66% yield). Analytical data were consistent with that of previously obtained samples.

### Method C:

To a 100-mL, 3-neck, round bottom flask were charged trimethylsulfoxonium bromide (0.560 g, 3.24 mmol) and NMP (5 mL). K₂CO₃ (1.073 g, 7.77 mmol) was added at less than 25° C., and the reaction was stirred at 20° C. for 1 h. 4-((6-(2-(2,4-Difluorophenyl)-1,1-difluoro-2-oxoethyl)pyridin-3-yl)oxy)benzonitrile (II) (1.0 g, 2.59 mmol) was added, and the reaction was stirred at 20° C. for 18 h, after which time HPLC analysis indicated that the reaction was incomplete. It was further stirred at 35° C. for 4 h, after which time HPLC analysis indicated that the starting material was consumed. 1H-1,2,4-Triazole (0.215, 3.11 mmol) was added, and the reaction was stirred at 20° C. for 18 h, at which point HPLC analysis indicated that the reaction was incomplete. It was further heated at 35° C. for 4 h, and cooled to 20° C. Water (20 mL) was added, and the reaction mixture was stirred for 30 min to afford a gummy precipitate, which was isolated by decanting off solvent. The crude product was purified by column chromatography (40 g silica, 0-50% EtOAc/hexanes over 10 min, hold for 15 min). Fractions containing pure product were concentrated to afford a white foam (0.89 g, 73% yield). Analytical data was consistent with that of previously obtained samples.

### Method D:

A 100-mL, 3-neck, round bottom flask was charged with trimethylsulfoxonium chloride (0.832 g, 6.48 mmol) and NMP (10 mL). K₂CO₃ (2.146 g, 15.554 mmol) was added at less than 25° C., and the reaction was stirred at 20° C. for 1 h. 4-((6-(2-(2,4-Difluorophenyl)-1,1-difluoro-2-oxoethyl)pyridin-3-yl)oxy)benzonitrile (II) (2.0 g, 5.18 mmol) was added, and the reaction was stirred at 20° C. for 18 h, after which time HPLC analysis indicated that the starting material was fully consumed. 1H-1,2,4-Triazole (0.43 g, 6.11 mmol) was added, and the reaction was stirred at 20° C. for 18 h, at which point HPLC analysis indicated that the reaction was complete. Water (25 mL) was added, and the reaction mixture was stirred for 30 min to afford a gummy precipitate, which was isolated by decanting off solvent. The crude product was purified by column chromatography (80 g silica, 0-50% EtOAc/hexanes over 10 min, hold for 15 min). Fractions containing pure product were concentrated to afford a white foam (1.5 g, 62% yield). Analytical data were consistent with that of previously obtained samples.

### Method E:

To a 250 mL jacketed reactor with the jacket set at 25° C. were added trimethylsulfoxonium bromide (6.16 g, 35.6 mmol), potassium carbonate (11.18 g, 81 mmol), and DMSO (37.5 mL). The slurry was stirred for 30 min then 4-((6-(2-(2,4-difluorophenyl)-1,1-difluoro-2-oxoethyl)pyridin-3-yl)oxy)benzonitrile (II) (12.5 g, 32.4 mmol) was added and the jacket was heated to 55° C. After 1 h, 1H-1,2,4-triazole (2.458 g, 35.6 mmol) was added and the mixture was stirred at 55° C. for 5 h. The jacket was turned down to 25° C. and 125 mL MTBE was added to the reaction then 125 mL water was added. The mixture was stirred vigorously for 30 min then was allowed to settle. The aqueous layer was removed and 125 mL water was added to the organic layer and the two were mixed for 15 min. 25 mL MTBE and 10 mL saturated brine were added and the layers mixed for 2 minutes then allowed to settle. The aqueous layer was removed from the reactor. The reactor was fitted with a distillation head and the jacket set to 65° C. 82 g of solvent were atmospherically distilled overhead (about 115 mL) then methanol (53 g, about 70 mL) was added. Distillation was continued until the overhead temperature was 65° C. and a total of 130 g of solvent had been distilled overhead (about 110 g MTBE and about 20 g MeOH; 33 g of methanol remained in the reactor). The jacket was cooled to 60° C. and water (3.4 g) was added dropwise. The mixture was then seeded with compound I. Additional water (3.2 g) was added slowly causing precipitation of more solids. The slurry was cooled to 20° C. over 4 h. After stirring at 20° C. for 1 h the solids were isolated by filtration and washing the reaction vessel with the mother liquor to clear out the solids. The solids were washed with 2:1 methanol/water w/w (2×10 mL). The solids were air dried to constant mass giving 4-((6-(2-(2,4-difluorophenyl)-1,1-difluoro-2-hydroxy-3-(1H-1,2,4-triazol-1-yl)propyl)pyridin-3-yl)oxy)benzonitrile (I) (10.08 g, 20.40 mmol, 63.0% yield) as a tan solid. Analytical data were consistent with that of previously obtained samples.

### Method F:

To a 250 mL jacketed reactor set at 25° C. were added trimethylsulfoxonium bromide (6.16 g, 35.6 mmol), potassium carbonate (11.18 g, 81 mmol), THF (62.6 mL), and water (12.51 mL). This slurry was stirred at 25° C. for 15 min and then 4-((6-(2-(2,4-difluorophenyl)-1,1-difluoro-2-oxoethyl)pyridin-3-yl)oxy)benzonitrile (II) (12.5 g, 32.4 mmol) was added and the mixture was stirred at 60° C. overnight. The jacket was cooled to 25° C., water (37.5 mL) was added and the layers mixed for 5 min. The aqueous layer was removed from the reactor. The organic layer was distilled atmospherically with jacket at 85° C. After 40 mL was distilled overhead, 37.5 mL DMSO was added. Distillation was continued with only 5 mL more solvent coming overhead. The jacket was cooled to 55° C. leaving about 20 mL THF in the reaction mixture. Potassium carbonate (11.18 g, 81 mmol) followed by 1H-1,2,4-triazole (2.458 g, 35.6 mmol) were added. The reaction was stirred at 55° C. for 5 h then MTBE (125 mL) and water (125 mL) were added and mixed for 15 min. The layers were separated. The organic layer was washed with a mixture of 125 mL water and 20 mL brine. The organic layer left in the jacketed reactor was distilled atmospherically. After 67 g of solvent was distilled overhead, 55.7 g methanol was added and distillation continued until 47 g more solvent had come overhead. The dark brown solution was cooled to 60° C. and then 3.02 g water was added slowly and the mixture was seeded. An additional 8.5 g water was added giving about 3:1 methanol/water w/w. The mixture was cooled to 20° C. over 2 h and the slurry was held at 20° C. overnight. The solids that formed were isolated by filtration, washing the reactor with the mother liquor. The solids were washed with 3:1 methanol/water w/w (20 g) and air dried to constant mass giving 4-((6-(2-(2,4-difluorophenyl)-1,1-difluoro-2-hydroxy-3-(1H-1,2,4-triazol-1-yl)propyl)pyridin-3-yl)oxy)benzonitrile (I) (11.62 g, 24.76 mmol, 77% yield) as a tan solid. ¹H NMR (400 MHz, DMSO-d₆) δ 8.47 (d, J=2.7 Hz, 1H), 8.36 (s, 1H), 7.99-7.89 (m, 2H), 7.71 (s, 1H), 7.69 (dd, J=8.7, 2.8 Hz, 1H), 7.51 (d, J=8.7 Hz, 1H), 7.30-7.19 (m, 3H), 7.13 (ddd, J=12.0, 9.2, 2.6 Hz, 1H), 7.05 (s, 1H), 6.88 (td, J=8.5, 2.6 Hz, 1H), 5.35 (d, J=14.6 Hz, 1H), 4.83 (d, J=14.6 Hz, 1H). ¹⁹F NMR (376 MHz, DMSO-d₆) δ −102.83 (td, J=22.5, 21.9, 9.2 Hz), −107.66 (dd, J=21.7, 13.5 Hz), −110.46 (d, J=9.4 Hz). ESIMS m/z 470.2 [(M+H)⁺].

The processes exemplified in Example 5 may be conducted at temperatures ranging from about −20° C. to about 100° C., or from about 20° C. to about 80° C.

Solvents that may be used in the processes exemplified in Example 5 may include at least one of dimethylsulfoxide (DMSO), dimethylformamide (DMF), tetrahydrofuran (THF), sulfolane, water, and N-methyl-2-pyrrolidone (NMP).

Bases that may be used in the processes exemplified in Example 5 may include metal carbonates such as, for example, potassium carbonate and sodium carbonate, metal alkoxides such as, for example, potassium tert-butoxide, or metal bicarbonates such as, for example, sodium and potassium bicarbonate.

What is claimed is:

1. A method of making a compound of Formula I 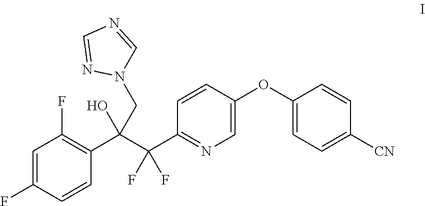 [SMILES: N#Cc1ccc(Oc2ccc(C(F)(F)C(O)(Cn3cncn3)c3ccc(F)cc3F)nc2)cc1]
comprising the step of contacting a compound of Formula II 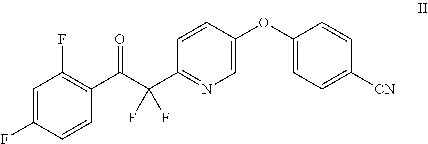 [SMILES: N#Cc1ccc(Oc2ccc(C(F)(F)C(=O)c3ccc(F)cc3F)nc2)cc1]
with a trialkylsulfoxonium halide, a base, and 1H-1,2,4-triazole.

2. The method of claim 1, wherein the trialkylsulfoxonium halide is one of trimethylsulfoxonium iodide, trimethylsulfoxonium bromide and trimethylsulfoxonium chloride.

3. The method of claim 1, wherein the base may be selected from the group including metal carbonates, metal alkoxides and metal bicarbonates.

4. The method of claim 1, wherein the base is potassium carbonate or sodium tert-butoxide.

5. The method of claim 1 further comprising a solvent selected from the group including dimethylsulfoxide (DMSO), dimethylformamide (DMF), sulfolane, tetrahydrofuran (THF), water, N-methyl-2-pyrrolidone (NMP), and mixtures thereof.

6. The method of claim 1 further comprising a solvent selected from the group including THF, water, DMSO, and mixtures thereof.

7. The method of claim 1 wherein the contacting is carried out from about −20° C. to about 100° C.

8. The method of claim 1 wherein the contacting is carried out from about 20° C. to about 80° C.

9. The method of claim 1, further comprising the step of:
contacting a compound of Formula III 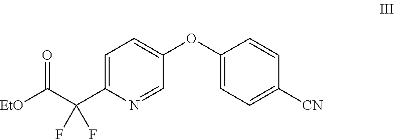 [SMILES: CCOC(=O)C(F)(F)c1ccc(Oc2ccc(C#N)cc2)cn1]
with a mixture formed by combining 1-bromo-2,4-difluorobenzene with a metal or an organometallic reagent, and
an acid,
to prepare the compound of Formula II.

10. The method of claim 9, further comprising an aprotic solvent selected from the group including diethyl ether, tetrahydrofuran, 1,2-dimethoxyethane, toluene, dioxane, methyl t-butyl ether, and mixtures thereof.

11. The method of claim 9 wherein the metal is magnesium and the organometallic reagent is an alkyllithium, or an alkylmagnesium halide.

12. The method of claim 11 wherein the alkyllithium is n-butyllithium, and the alkylmagnesium halide is isopropylmagnesium chloride.

13. The method of claim 9, wherein the contacting is carried out between about −80° C. and about 50° C.

14. The method of claim 9, wherein the acid is selected from the group including HCl, HBr, H₂SO₄, H₃PO₄, HNO₃, acetic acid, and trifluoroacetic acid.

15. The method of claim 9, further comprising the step of:
contacting a compound of Formula IV 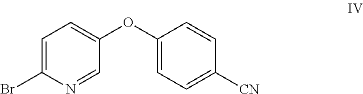 [SMILES: N#Cc1ccc(Oc2ccc(Br)nc2)cc1]
with ethyl 2-bromo-2,2-difluoroacetate and a metal to prepare the compound of Formula III.

16. The method of claim 15, wherein the metal is copper.

17. The method of claim 15, further comprising a solvent selected from the group including DMSO, DMF, THF, NMP, and mixtures thereof.

18. The method of claim 15 wherein the contacting is carried out between about room temperature and about 100° C.

19. The method of claim 15, further comprising the step of:
contacting a compound of Formula V 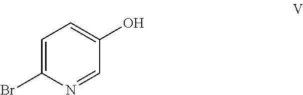
with 4-fluorobenzonitrile or 4-nitrobenzonitrile, and a base to prepare the compound of Formula IV.

20. The method of claim 19 wherein the base is selected from cesium carbonate and potassium carbonate.

21. The method of claim 19, wherein the step of contacting the compound of Formula V with 4-fluorobenzonitrile or 4-nitrobenzonitrile, and a base further includes a solvent.

22. The method of claim 21, wherein the solvent is selected from the group including dimethyl sulfoxide, N,N-dimethylacetamide, N,N-dimethylformamide, N-methyl-2-pyrrolidone, and mixtures thereof.

23. The method of claim 19 wherein the step of contacting the compound of Formula V with 4-fluorobenzonitrile or 4-nitrobenzonitrile, and a base is carried out between about room temperature and about 120° C.

24. The method of claim 19, further comprising the step of:
contacting a compound of Formula VI 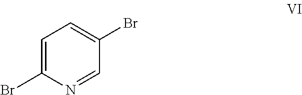
with a magnesium-halogen exchange reagent, a borate, and an oxidizing agent to prepare the compound of Formula V.

25. The method of claim 24, wherein the magnesium-halogen exchange reagent is iso-propylmagnesium chloride.

26. The method of claim 24, wherein the borate is selected from the group including B(OMe)₃, B(OEt)₃ and B(Oi-Pr)₃.

27. The method of claim 24, wherein the oxidizing agent is selected from the group including hydrogen peroxide, peracetic acid, and a mixture of hydrogen peroxide and acetic acid.

28. The method of claim 24, further comprising a solvent selected from the group including THF, 2-methyltetrahydrofuran, methyl t-butyl ether, dioxane, and mixtures thereof.
